(19) United States
(12) Patent Application Publication
(10) Pub. No.: US 2026/0027066 A1
(43) Pub. Date: Jan. 29, 2026

(54) NANOSCALE MATRIMERES

(71) Applicant: THE BOARD OF TRUSTEES OF THE UNIVERSITY OF ILLINOIS, Urbana, IL (US)

(72) Inventors: Jae-Won SHIN, Chicago, IL (US); Koushik DEBNATH, Chicago, IL (US)

(21) Appl. No.: 18/997,124

(22) PCT Filed: Jul. 24, 2023
(86) PCT No.: PCT/US2023/070806

§ 371 Date: Jan. 20, 2025

Related U.S. Application Data
(60) Provisional application No. 63/391,613, filed on Jul. 22, 2022.

(51) Int. Cl. A61K 9/51; A61K 9/00; A61K 9/12; A61K 31/7088; A61K 33/42; A61P 11/00

(52) U.S. Cl. CPC A61K 9/5169; A61K 9/0019; A61K 9/0073; A61K 9/12; A61K 31/7088; A61K 33/42; A61P 11/00

(57) ABSTRACT

Disclosed are isolated nanoparticles composed of an extracellular matrix which comprises at least one heparin binding domain and nucleic acid or polyphosphate, as well as compositions comprising the nanoparticles and methods of treating tissue Injury.

### INTRODUCTION

This application claims the benefit of priority from U.S. Provisional Application Ser. No. 63/391,613, filed Jul. 22, 2022, the contents of which are incorporated herein by reference in their entireties.

This invention was made with government support under grant no. HL141255 awarded by the National Institutes of Health. The government has certain rights in this invention.

### REFERENCE TO AN ELECTRONIC SEQUENCE LISTING

The contents of the electronic sequence listing (name: UIC0104WO_ST26.xml; size: 70,145 bytes; and date of creation: Jul. 18, 2023) is herein incorporated by reference in its entirety.

### BACKGROUND

Vascular hyperpermeability remains the major cause of tissue damage after trauma. Compromised endothelial barrier function leads to inflammation and uncontrolled influx of proteinaceous fluid into the interstitium, a hallmark of edema. Treatment of vascular hyperpermeability remains a general challenge for vascularized organs after injury, including the lungs. Restoration of endothelial barrier: requires endothelial cell proliferation, followed by formation of adherens junctions, which are primarily mediated by VE-cadherin. Importantly, stabilization of adherens junctions is known to require key components of cell-extracellular matrix (ECM) mechanotransduction, including β1 and β3 integrins that bind to Arg-Gly-Asp (RGD), and focal adhesion kinase (FAK), wherein downregulating these components in endothelial cells results in persistent barrier leakage. After injury, fibroblasts in the interstitium synthesize collagenous ECM fibers to replace the damaged ECM network, which is followed by gradual remodeling of the ECM to restore normal tissue. However, this process can become aberrant with persistent edema, since interstitial pressure can increase the activation of fibroblasts to produce excess collagen and to promote ECM crosslinking, thereby resulting in fibrosis with impaired tissue mechanics. Thus, there is a need to rapidly restore endothelial-ECM interactions in order to reverse vascular hyperpermeability after injury and to promote normal tissue remodeling.

### SUMMARY OF THE INVENTION

This invention is an isolated nanoparticle composed of at least one extracellular matrix protein or fragment thereof, the at least one extracellular matrix protein or fragment thereof including at least one heparin binding domain, and optionally at least one cell adhesion sequence; and nucleic acid molecules or polyphosphate molecules composed of about 30 to about 10000 nucleotides or phosphate units. In one aspect, the at least one extracellular matrix protein is fibronectin, fibrinogen/fibrin, vitronectin, laminin, collagen, fibulin, fibrillin, or tropoelastin, or a combination thereof. In another aspect, the nucleic acid molecules include fragmented genomic DNA from mammalian cells. In another aspect, the nanoparticle is isolated from plasma or from cell culture medium. In other aspects, the nanoparticles are reconstituted from: at least one purified or recombinant extracellular matrix protein and purified nucleic acid molecules from mammalian cells; at least one purified or recombinant extracellular matrix protein and synthetic nucleic acid molecules; at least one purified or recombinant extracellular matrix protein and polyphosphate molecules; at least one synthetic extracellular matrix-derived peptide and purified nucleic acid molecules from mammalian cells; at least one synthetic extracellular matrix-derived peptide and synthetic nucleic acid molecules; and/or at least one synthetic extracellular matrix-derived peptide and polyphosphate molecules.

In one aspect, this invention is a composition including a pharmaceutically acceptable excipient or diluent and an isolated nanoparticle composed of at least one extracellular matrix protein or fragment thereof, the at least one extracellular matrix protein or fragment thereof including at least one heparin binding domain; and nucleic acid molecules or polyphosphate molecules composed of about 30 to about 10000 nucleotides or phosphate units.

In a further aspect, this invention provides a method of enhancing or restoring endothelial barrier function in a subject by administering to a subject an effective amount of the isolated nanoparticle of this invention thereby enhancing or restoring endothelial barrier function in the subject.

In a still further aspect, this invention provides a method of treating tissue injury in a subject by administering to the subject an effective amount of the isolated nanoparticle of this invention thereby treating tissue injury in the subject, wherein the tissue injury optionally includes vascular hyperpermeability and/or edema; or tissue damage, inflammation, degeneration, and/or fibrosis.

Further provided by this invention is a method for preparing the nanoparticle of this invention by contacting, at a pH in the range of about 4 to 6, the at least one extracellular matrix protein or fragment thereof with the nucleic acid molecules or polyphosphate molecules thereby preparing the nanoparticle.

### BRIEF DESCRIPTION OF THE DRAWINGS

FIGS. 1A-1C show the presence of fibronectin positive (FN⁺) matrimere subpopulation distinct from CD63⁺ vesicles. (FIG. 1A) Quantification of mouse mesenchymal stem cell (MSC)-secreted extracellular nanostructure subpopulations. The pellet from ultracentrifugation (100,000 g) was resuspended and contacted with anti-CD63 or anti-FN antibody attached to either magnetic particles (MP) or polyethylene-glycol (PEG)-based hydrogels to deplete a specific subpopulation. Nanoparticle tracking analysis was used to count particles before and after depletion. Shown is the quantification of particles depleted by anti-FN or anti-CD63 antibody compared to negative control. n=3 experiments, ****p<0.0001. (FIG. 1B) Percentage of the TRITON™ X sensitive fraction before (CTRL) and after negative selection with FN or CD63 antibody. n=3 experiments, **p<0.01, ***p<0.001. (FIG. 1C) Confirmation of nanostructures after positive selection with FN or CD63 antibody. Circular equivalent diameter (CED) and solidity. n=24 for CD63⁺ and n=38 for FN⁺ nanostructures. p-values were derived from one-way ANOVA followed by Tukey's post-test. All data are shown as mean±s.d.

FIGS. 2A-2C show the presence of FN⁺ particles in mouse blood plasma. (FIG. 2A) Total number of nanostructures in plasma before (ctrl) and 8 hours after lipopolysaccharide (LPS; 10 mg/kg, i.p.) treatment. ***p<0.001. (FIG. 2B) Percentage of FN⁺ particles in blood plasma. *p<0.05. (FIG. 2C) Total number of FN⁺ particles per μl plasma. n=3 experiments. p-values were derived from unpaired T-test. All data are shown as mean #s.d.

FIGS. 3A-3B show that MSC-secreted FN⁺ matrimeres restore LPS-induced endothelial permeability in vitro. (FIG. 3A) TEER kinetics of the human umbilical vein endothelial cell (HUVEC) monolayer showing the rescue of LPS (1 μM)—compromised TEER by the CD63-depleted fraction of MSC-secreted nanostructures. (FIG. 3B) Changes in TEER from 6.5 hours to 24 hours after washing out LPS followed by treatment. ****p<0.0001. n=3 experiments. p-values were derived from one-way ANOVA followed by Dunnett's T3 post-test. All data are shown as mean±s.d.

FIGS. 4A-4B show that FN⁺ matrimeres reverse LPS-induced lung edema and vascular hyperpermeability in vivo. (FIG. 4A) Lung edema by quantifying lung wet-dry ratio. (FIG. 4B) Vascular permeability by quantifying Evans blue albumin (EBA) accumulation. ****p<0.0001, ***p<0.001, **p<0.01. n=4 mice for each experimental group. p-values were derived from one-way Welch's ANOVA followed by Dunnett's T3 post-test. All data are shown as mean±s.d.

FIGS. 5A-5D show that DNA is required for the ability of FN-enriched fraction from MSCs to reverse lung edema and vascular permeability in vivo. (FIG. 5A) Quantification of total DNA per 10¹⁰ particles with or without depletion of nanostructures with FN or CD63 antibody. n=3 experiments. (FIG. 5B) Quantification of FN⁺ particles secreted from MSCs collected after treatment of the collected medium with DNase I. n=3 technical replicates. Quantification of endothelial barrier function in the lungs in vivo as determined by wet-dry ratio of lung tissue (FIG. 5C) and Evans blue albumin (EBA) accumulation (FIG. 5D). ****p<0.0001, ***p<0.001, **p<0.01, *p<0.05. n=4 mice for each experimental group. p-values were derived from one-way Welch's ANOVA followed by Dunnett's T3 post-test. All data are shown as mean±s.d.

FIGS. 6A-6C show the effects of pharmacological modulators on FN⁺ matrimere secretion. (FIG. 6A) Overview of the potential biogenesis pathway for FN⁺ matrimeres, known regulators, and their pharmacological modulators. (FIG. 6B) Total number of nanostructures per MSC after 24 hours in culture with drugs or DMSO control. (FIG. 6C) Percent of FN⁺ particles in MSC-secreted nanostructures. (FIG. 6D) Total number of FN⁺ particles per cell. *p<0.05. n=3 experiments. p-values from one-way ANOVA followed by Tukey's post-test. All data are shown as mean±s.d. BMS-P5 (CAS No. 1550371-22-6); Bafilomycin-A1 (BafA1, CAS No. 88899-55-2); curcumin analog C1 (CA-C1, CAS No. 39777-61-2); ML-SA5 (CAS No. 2418670-70-7).

FIGS. 7A-7D show that matrimeres can be reconstituted in vitro. (FIG. 7A) Reconstitution of FN-DNA particles using genomic DNA fragments from MSCs. Shown is pH-dependent particle formation from FN and fragmented DNA at 1:1 w/v. (FIGS. 7B-7C) TEM analysis of FN-DNA matrimeres. Quantification of CED (FIG. 7B) and (FIG. 7C) solidity. n=501 particles from 10 images pooled from 3 different batches. (FIG. 7D) Stability of reconstituted matrimeres at neutral pH in vitro. The data points were fitted to one-phase decay curves. t1/2 and plateau for FN-DNA: 0.94, 89.4 h; Vitronectin (VTN)-DNA: 0.92, 10.3 h; FN-polyphosphate (polyp): 0.46, 4.0 h. n=3 batches for each group.

FIG. 8A-8B show the effects of reconstituted FN-DNA matrimeres on the recovery of TEER after 6 hours LPS (1 μM) treatment in vitro. (FIG. 8A) TEER kinetics. (FIG. 8B) Percentage of TEER recovery. n=3 experiments.

FIGS. 9A-9C show that FN-DNA particles reverse LPS-induced lung edema and vascular hyperpermeability in vivo. (FIG. 9A) Biodistribution of FN-DNA matrimeres in lungs. Mice were treated with LPS (7.5 mg/kg) for 4 hour, followed by delivery of Cy7-conjugated FN-DNA matrimeres via the i.t. route and IVIS imaging at different time points. Radiance values from Cy7 signals were normalized to the mean value of the data points from t=0.5 hour in lung tissue. The data points were fitted to one-phase decay curves. t1/2 and plateau for lung: 2.1 h, 0.47. n=4 mice for each time point. (FIG. 9B) Lung edema by quantifying lung wet-dry ratio. (FIG. 9C) Lung vascular permeability by quantifying Evans blue albumin (EBA) accumulation. Soluble FN (sFN) and VTN (SVTN) protein groups (10 μg/ml, 30 μl per 20 g mouse) were included as controls. The doses are indicated in the unit of 10⁸ per 20 g mouse. n=4 mice for each group. *p<0.05, **p<0.01, ***p<0.001, ****p<0.0001 via one-way ANOVA with Tukey's multiple comparisons test for FIG. 9A and via Welch's one-way ANOVA with Dunnett's T3 post-test for FIGS. 9B-9C and All data are shown as mean±s.d.

FIG. 10 shows the design of FN-derived peptides. Proposed peptide sequences containing key domains are shown in the context of full-length FN sequence. PPSRN (SEQ ID NO: 1); RGDSP (SEQ ID NO:2); PPRRARVT (SEQ ID NO:3).

### DETAILED DESCRIPTION OF THE INVENTION

A novel class of nanoparticles, also referred to herein as “matrix exomeres” or “matrimeres,” have been developed that are composed of at least one type of extracellular matrix protein and nucleic acid molecules and/or polyphosphate molecules. The nanoparticles have been shown to improve endothelial functions and reverse edema and vascular leakage following tissue injury.

Accordingly, the present invention provides an isolated, non-vesicular nanoparticle composed of at least one (e.g., 2, 3, 4, 5, 6, 7, 8, 9 or 10) extracellular membrane (ECM) protein or a fragment thereof and nucleic acid molecules or polyphosphate molecules. As used herein the term “isolated” is meant to describe a nanoparticle that is in an environment different from that in which the element naturally occurs. Similarly, an isolated protein or nucleic acid molecule refers to a protein or nucleic acid molecule that is in an environment different from that in which the element naturally occurs. In some aspects, a nanoparticle, protein, or nucleic acid molecule of the invention is purified. “Purified” as used herein refers to a nanoparticle, protein, or nucleic acid molecule removed from an environment in which it was produced and is at least 60% free, preferably 75% free, and most preferably 90% free from other components with which it is naturally associated.

The term “extracellular matrix” refers to a protein-rich substance that is found in between cells in animal or human tissue and serves as a structural element in tissues. It typically includes a complex mixture of polysaccharides and proteins secreted by cells. An extracellular matrix protein is a protein found in the extracellular matrix of a mammalian cell. An extracellular matrix protein of this invention has an amino acid sequence that includes a heparin binding domain. Heparin binding domains of extracellular matrix proteins have been shown to contain the consensus sequence motifs BBXB and/or BBBXXB, where B is independently lysine, arginine, ornithine, or histidine and each X is independently a naturally occurring amino acid. The capacity of an extracellular matrix protein or fragment thereof to bind to heparin can be assessed by any suitable binding assay. See, e.g., Example 1.

In some aspects, the nanoparticle of the invention includes at least one extracellular matrix protein, wherein the at least one extracellular matrix protein includes at least one (e.g., 2, 3, 4, 5, 6, 7, 8, 9 or 10) heparin binding domain. Examples of extracellular matrix proteins and the amino acid sequence of the heparin binding domain(s) thereof, which may be included in the nanoparticle of the invention, are listed in Table 1.

TABLE 1

| | GENBANK | SEQ | | SEQ |
|---|---|---|---|---|
| | Accession | ID | Heparin Binding | ID |
| ECM Protein | No. | NO: | Domain | NO: |
| Fibronectin | NP_997647.2 | 4 | WQPPRARI | 5 |
| | | | PPRARV | 6 |
| | | | PRRGRV | 7 |
| Fibrinogen/ | NP_005132.2 | 8 | GHRPLDKKREEAPSL | 9 |
| Fibrin | | | RPAPPPISGGGYR | |
| Vitronectin | NP_000629.3 | 10 | KKQRFRHRNRKG^{a} | 11 |
| Laminin | NP_005550.2 | 12 | KEGYKVRLDLNITLE | 13 |
| subunit α-1 | | | FRTTSK^{b} | |
| | | | KATPMLKMRTSFHGCIK^{b} | 14 |
| | | | IKLLI^{b} | 15 |
| | | | KDFLSIELVRGRVK^{b} | 16 |
| | | | PPFLMLLKGSTRENKTKTF | 18 |
| | | | R^{c} | |
| Laminin | KAI4045787.1 | 17 | RLVFALGTDGKKLRIK | 19 |
| subunit α-3 | | | SKEKCNDGK^{c} | |
| Laminin | KAI4019510.1 | 20 | PLFLLHKKGKNLSKP | 21 |
| subunit α-4 | | | KASQNKKGGKSK^{c} | |
| | | | TLFLAHGRLVYMEN | 22 |
| | | | VGHKKLKIR^{c} | |
| Laminin | KAI4006273.1 | 23 | TPGLGPRGLQATARKAS | 24 |
| subunit α-5 | | | RRSRQPARHPACML^{c} | |
| | | | RQRSRPGRWHKVSVRWE | 25 |
| | | | KNR^{c} | |
| Collagen | NP_000079.2 | 26 | KGHRGF^{d} | 27 |
| | KAI4014616.1 | 28 | KGIRGH^{d} | 29 |
| Fibulin-3 | NP_001034437.1 | 30 | Gln18-Phe493^{e} | 31 |
| Fibulin-4 | NP_058634.4 | 32 | Gln28-Phe443^{e} | 33 |
| Fibulin-5 | NP_006320.2 | 34 | Gln24-Phe448^{e} | 35 |
| Fibrillin | BAD16739.1 | 36 | Asp1070-Thr1237^{f} | 37 |
| | | | Asp1689-Glu1807^{f} | 38 |
| Tropoelastin | AAC98394 | 39 | Gly27-Lys724^{g} | 40 |

^{a}Wilkins et al. (2003) Cell Commun. Adhes. 10:85-103
^{b}Edgar & Thoenen (1984) EMBO J. 3: 1463-8; Skubitz et al. (1991) J. Cell. Biol. 115:1137-48; Tashiro et al. (1999) Biochem J. 340 (Pt 1):119-26; Yoshida et al. (1999) J. Cell Physiol. 179:18-28.
^{c}Ishihara et al. (2018) Nat. Commun. 9: 2163.
^{d}San Antonio et al. (1994) J. Cell Biol. 125:1179-1188.
^{e}Djokic et al. (2013) J. Biol. Chem. 288 (31):22821-22835.
^{f}Cain et al. (2005) J. Biol. Chem. 280 (34):30526-30537.
^{g}Tu & Weiss (2010) Matrix Biol. 29 (2):152-159.

In some aspects, the at least one extracellular matrix protein of the nanoparticle is human fibronectin, human fibrinogen/fibrin, human vitronectin, human laminin, human collagen, human fibulin, human fibrillin, human tropoelastin, as well as orthologs, or combinations thereof. In some aspects, a nanoparticle of the invention is composed of a single type of extracellular protein, e.g., fibronectin. In other aspects, a nanoparticle of the invention is composed of multiple types of extracellular proteins. By way of illustration, the nanoparticle of the invention may be composed of human fibronectin and human laminin; human fibronectin and mouse laminin; human fibronectin, mouse laminin, and bovine collagen; mouse fibronectin, and bovine collagen; etc. as the extracellular matrix proteins. In this respect, the extracellular matrix protein of the nanoparticle of the invention may be an extracellular matrix protein that shares 80%, 85%, 90%, 95%, 97% or 99% identity with any one of the sequences in Table 1. In some aspects, the at least one extracellular matrix protein is human fibronectin or an ortholog thereof. In some aspects, the at least one extracellular matrix protein is a combination of proteins, e. g., extracellular matrix proteins isolated from the extracellular matrix of a cell.

In some aspects, the at least one extracellular matrix protein of the nanoparticle is a fragment of a full-length extracellular matrix protein (e.g., the extracellular matrix proteins of Table 1). A “fragment” of a sequence may typically be, for example, a shorter portion of the full-length sequence of a nucleic acid molecule or amino acid sequence. Thus, a fragment is typically composed of a sequence that is identical to the corresponding stretch within the full-length sequence. The fragment of a sequence of the present invention is composed of a continuous series of entities such as nucleotides or amino acids corresponding to the continuous series of entities in the molecule from which the fragment is derived. A “fragment” of a protein may include an N-terminal and/or C-terminal truncation so long as the fragment retains a heparin binding domain. In some aspects, a fragment of an extracellular domain is an extracellular matrix-derived fragment or peptide having a length of at least 20, 25, 30, 35, 40, 45, 50, 55, 60, 65, 70, 75, 80, 85, 90, 95, 100, 120, 140, 160, 180, 200, 300 or 400 amino acid residues, and includes at least one heparin binding domain.

In other aspects, the at least one extracellular matrix protein or fragment thereof, in addition to including at least one heparin binding domain, further includes at least one cell adhesion sequence. Examples of suitable cell adhesion sequences include RGD or RGD-containing sequences such as RGDS (SEQ ID NO: 41), RGDSP (SEQ ID NO: 42), RGDSPK (SEQ ID NO:43), RGDTP (SEQ ID NO:44), RGDSPASSKP (SEQ ID NO: 45), or GGGGRGDSP (SEQ ID NO: 46) derived from fibronectin; KQAGDV (SEQ ID NO: 47), PHSRN (SEQ ID NO:48), PPSRN (SEQ ID NO: 49), YIGSR (SEQ ID NO: 50) or RLVSYNGIIFFLK (SEQ ID NO: 51) derived from laminin; DGEA (SEQ ID NO: 52) derived from collagen; or VAPG (SEQ ID NO: 53) derived from elastin.

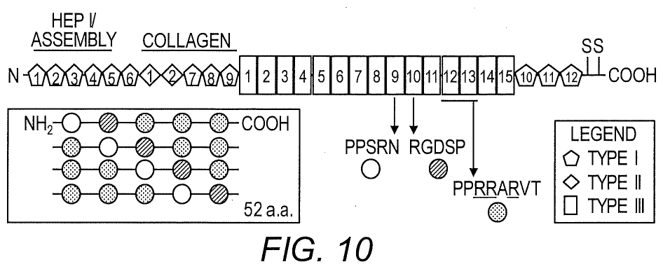

An extracellular matrix protein or fragment thereof may be isolated from a natural source (e.g., cells), prepared by conventional recombinant or synthetic methods, and/or purchased from commercial sources. In some aspects, an extracellular matrix protein or fragment thereof is derived or isolated from cells, e.g., stem cells such as mesenchymal stem cells. When prepared by recombinant or synthetic methods, the at least one extracellular matrix protein or fragment/peptide thereof may include one or more heparin binding domains and optionally one or more cell adhesion sequences, wherein the one or more heparin binding domains and optionally one or more cell adhesion sequences are linked together by linkers, e.g., glycine or glycine-serine linkers such as GGG or GGS. See, e.g., FIG. 10.

Recombinant production of extracellular matrix protein or fragment/peptide thereof may take place in host cell lines, e.g., Chinese hamster ovary host cell lines (CHO-host cell lines). CHO-host cell lines have been approved by the Food and Drug Administration (FDA) for the recombinant production of protein therapeutics due to their high safety level. For clinical studies the production of the recombinantly produced, preferably human, protein can further be adjusted to good manufacturing practice (GMP) conditions.

When isolated, an extracellular matrix protein or fragment/peptide thereof of use in this invention may include or be free of one or more proteins that may alter the activity of the nanoparticle. Thus, in some aspects, the at least one extracellular matrix protein or fragment/peptide thereof may include 0%, 0.1%, 0.5%, 1%, 5%, or 10% by weight of one or more of cytoplasmic domain-44 (CD44), hyaluronan, syndecan, basic fibroblast growth factor (bFGF), hepatocyte growth factor (HGF), platelet-derived growth factor (PDGF), vascular endothelial growth factor (VEGF), tenascin, chondroitin sulfate B, integrins, decorin, and/or transforming growth factor beta (TGF beta). In some aspects, the nanoparticle includes an integrin.

As used herein, the term “nucleic acid molecules” or “polynucleotides” refer to DNA or RNA molecules. In some aspects, the nucleic acid molecules are polymers comprising or consisting of nucleotide monomers covalently linked to each other by a phosphodiester bond of the sugar/phosphate backbone. The term “nucleic acid molecules” also includes modified nucleic acid molecules such as, for example, DNA or RNA molecules that are base modified, sugar modified, or backbone modified. Exemplary nucleic acid molecules of use in the invention include, but are not limited to fragmented genomic DNA, mitochondrial DNA, circular DNA, or RNA such as mRNA and miRNA.

In some aspects, the nucleic acid molecules are DNA, which may optionally be single-stranded or double-stranded. In the double-stranded form, the first strand nucleotides typically hybridize to the second strand nucleotides by, for example, A/T base pairing and G/C base pairing. In some aspects, the nucleic acid molecule has a GC content of 40%, 50%, or 60%. In some aspects, the nucleic acid molecules are nucleic acid molecules from cells. In some aspects, the nucleic acid molecules are nucleic acid molecules mammalian cells, bacterial cells, and/or fungal cells. In some aspects, the nucleic acid molecules are nucleic acid molecules isolated from mammalian cells and optionally purified. In some aspects, the nucleic acid molecules are genomic DNA from mammalian cells, e.g., mesenchymal stem cells. In some aspects, the nucleic acid molecules are genomic DNA isolated from cells (e.g., mammalian cells) and optionally purified. In other aspects, the nucleic acid molecules are synthetic nucleic acid molecules, e.g., synthetic or recombinant DNA molecules. In some aspects, the nucleic acid molecules are genomic DNA that have been fragmented (e.g., by sonication, restriction enzymes and the like).

“Polyphosphate” refers to refers to a chain of phosphate groups and may include alternating sugar and phosphate groups, e.g., as in the sugar-phosphate backbone of nucleic acid molecules. In some aspects, “polyphosphate” refers to a molecule having the structure of Formula I:

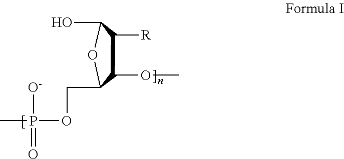

where R is H or OH and n is an integer of 2 or more. In some aspects, a polyphosphate may comprise a polyphosphate of Formula I grafted to other polymers or consist of or consist essentially of a polyphosphate of Formula I.

In some aspects, the nucleic acid molecules and/or polyphosphate molecules of the nanoparticle of the invention are composed of about 30 to about 10000 nucleotides (i.e., n of Formula I is an integer in the range of 30 to 10000). In some aspects, the nucleic acid molecules are genomic DNA from mammalian cells that have been fragmented to have a length in the range of 30 to 10000 nucleotides (e.g., 30, 40, 50, 60, 70, 80, 90, 100, 200, 250, 300, 350, 400, 450, 500, 750, 1000, 1250, 1500, 2000, 2500, 5000, 7500 or 10000 nucleotides, or any range delimited thereby). In some aspects, the nucleic acid molecules and/or polyphosphate molecules of the nanoparticle of the invention are composed of about 250 to about. 1500, or about 500 to about 1000 nucleotides or phosphate units.

In some aspects, the nanoparticle is composed of only nucleic acid molecules or polyphosphate molecules. In other aspects, the nanoparticle is composed of nucleic acid molecules and polyphosphate molecules. In other aspects, the nanoparticle is composed of nucleic acid molecules from a single source, e.g., a homogenous mixture of natural nucleic acids molecules of about 30 to about 10000 nucleotides in length (e.g., 250 to 1500 nucleotides in length), or a homogenous population of synthetic or artificial nucleic acid molecules having the same nucleotide sequence that is about 30 to about 10000 nucleotides in length (e.g., 250 to 1500 nucleotides in length).

As described herein, the nanoparticle of this invention may be isolated from plasma; isolated from the medium of cultured cells, i.e., the nanoparticles are secretions of cultured cells such as mesenchymal stem cells; or reconstituted by combining the components in vitro, i.e., combining at least one extracellular matrix protein or fragment thereof with nucleic acid molecules or polyphosphate molecules. Reconstituted nanoparticles may be produced from at least one isolated or recombinant extracellular matrix protein and isolated nucleic acid molecules from mammalian cells (e.g., fragmented genomic DNA, mitochondrial DNA, or RNA such as mRNA, miRNA, etc.); at least one isolated or recombinant extracellular matrix protein and synthetic nucleic acid molecules; at least one isolated or recombinant extracellular matrix protein and polyphosphate molecules; at least one synthetic extracellular matrix-derived peptide and isolated nucleic acid molecules from mammalian cells (e.g., fragmented genomic DNA, mitochondrial DNA, or RNA such as mRNA, miRNA, etc.); at least one synthetic extracellular matrix-derived peptide and synthetic nucleic acid molecules; or at least one synthetic extracellular matrix-derived peptide and polyphosphate molecules.

A reconstituted nanoparticle of the invention may be prepared by contacting, at a pH in the range of about 4 to 6, the at least one extracellular matrix protein or fragment thereof with the nucleic acid molecules or polyphosphate molecules thereby preparing the nanoparticle. In some aspects, the at least one extracellular matrix protein or fragment thereof is contacted with the nucleic acid molecules or polyphosphate molecules at a pH of at least 4, but not greater than 6. In some aspects, the pH used in the preparation of the nanoparticle is at about 4, 4.5, 5, 5.5 or 6, preferably 5 or 5.5.

A nanoparticle from plasma, medium of cultured cells or reconstituted as described herein may be isolated by various methods including, but not limited to, differential or serial centrifugation and/or immunoaffinity-based separation as described herein, e.g., using anti-CD63 antibodies to remove CD63+ extracellular vesicles.

In comparison to exosomes or other particles produced by cells, the nanoparticle of the present invention does not include a membrane and is therefore a non-vesicular nanoparticle. The nanoparticle of the invention has a particle size in the range of about 50 to about 200 nm, with a mean particle size of about 100 nm.

Having demonstrated that a nanoparticle of this invention restores endothelial barrier function after injury, the present invention also provides methods of enhancing or restoring endothelial barrier function and treating tissue injury in a subject by administering to the subject a nanoparticle of this invention or a composition containing the same. In some aspects, the nanoparticle of the invention is administered to improve endothelial functions and reverse edema and/or vascular leakage in a subject. In other aspects, the nanoparticle of the invention is administered to treat tissue damage, degeneration, fibrosis, or a combination thereof. Vascular hyperpermeability due to ischemic and traumatic injuries is linked to edema, aging, inflammation, tissue fibrosis, hemorrhagic shock, sepsis, and burns. Thus, the methods of this invention may be used in the ameliorating or treating any one or more of these conditions.

The As used herein, “subject” refers to an individual. Thus, subjects include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject is optionally a mammal such as a primate or a human.

Although not precluded, treating a disease or condition does not require that the disease, condition, or symptoms associated therewith be completely eliminated, including the treatment of acute or chronic signs, symptoms and/or malfunctions. “Treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. “Treatment” therefore also includes relapse prophylaxis or phase prophylaxis. The term “treat” and synonyms contemplate administering a therapeutically effective amount of the nanoparticle of the invention to an individual in need of such treatment. A treatment can be orientated symptomatically, for example, to suppress symptoms. Treatment can be carried out over a short period, be oriented over a medium term, or can be a long-term treatment, for example within the context of a maintenance therapy.

The nanoparticle may be administered by engraftment, wherein the cells are injected into the subject, for example, intravenously, intratracheally, intra-muscularly, intra-arterially, intra-bone, via inhalation, and the like. In certain embodiments, administration involves engrafting about 10², 10⁴, 10⁶, 10⁷, 10⁸, 10⁹, 10¹⁰, 10¹², or more nanoparticles. The number of nanoparticles engrafted may be chosen based on the route of administration and/or the severity of the condition for which the nanoparticles are being engrafted.

Compositions containing the nanoparticles can be prepared by combining the nanoparticles with a pharmaceutically acceptable carrier or diluent. The phrase “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and the like. The use of such media and agents for pharmaceutically active substances is well known in the art. Except insofar as any conventional media or agent is incompatible with the nanoparticles of the present disclosure, its use in therapeutic compositions is contemplated. Pharmaceutical compositions can be determined by one skilled in the art depending upon, for example, the intended route of administration, delivery format and desired dosage. See, for example, Remington's Pharmaceutical Sciences, 18th Edition, (A. R. Gennaro, ed.), 1990, Mack Publishing Company.

The compositions of the invention can be incorporated in an injectable formulation. The formulation may also include the necessary physiologically acceptable carrier material, excipient, lubricant, buffer, surfactant, antibacterial, bulking agent (such as mannitol), antioxidants (ascorbic acid or sodium bisulfite) and the like.

Acceptable formulation materials preferably are nontoxic to recipients at the dosages and concentrations employed. The pharmaceutical composition may contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials may include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA; complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as PLURONICS™, PEG, sorbitan esters, polysorbates such as polysorbate 20 and polysorbate 80, TRITON™, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, Remington's Pharmaceutical Sciences, Id.

The primary vehicle or carrier in a pharmaceutical composition may be either aqueous or nonaqueous in nature. For example, a suitable vehicle or carrier may be water for injection, physiological saline solution, or artificial cerebrospinal fluid, possibly supplemented with other materials common in compositions for parenteral administration. Neutral buffered saline or saline mixed with serum albumin are further exemplary vehicles. Pharmaceutical compositions can comprise Tris buffer of about pH 7.0-8.5, or acetate buffer of about pH 4.0-5.5, or MES buffer of about 6.5-7.0, which may further include sorbitol or a suitable substitute therefore. Pharmaceutical compositions of the invention may be prepared for storage by mixing the selected composition having the desired degree of purity with optional formulation agents (Remington's Pharmaceutical Sciences, Id.) in the form of a lyophilized cake or an aqueous solution.

The nanoparticles or composition can be provided by sustained release systems, by encapsulation or by implantation devices. The compositions may be administered by bolus injection or continuously by infusion, or by implantation device. The composition also can be administered locally via implantation of a membrane, sponge, or another appropriate material onto which the nanoparticles have been absorbed or encapsulated. Where an implantation device is used, the device may be implanted into any suitable tissue or organ. The injections may be given as a one-time treatment, repeated (daily, weekly, monthly, annually etc.) in order to achieve the desired therapeutic effect.

The compositions of the invention can be delivered parenterally. When parenteral administration is contemplated, the therapeutic compositions for use in this invention may be in the form of a pyrogen-free, parenterally acceptable aqueous solution. A particularly suitable vehicle for parenteral injection is sterile distilled water. Preparation can involve the formulation with an agent, such as injectable microspheres, bio-erodible particles, polymeric compounds (such as polylactic acid or polyglycolic acid), beads or liposomes, that may provide controlled or sustained release of the nanoparticles, which may then be delivered via a depot injection. Formulation with hyaluronic acid has the effect of promoting sustained duration in the circulation. Implantable drug delivery devices may be used to introduce the desired composition.

These compositions may also contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like.

Supplementary active ingredients also can be incorporated into the compositions. The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intraperitoneal, or intravenous injection. Such compositions would normally be administered as pharmaceutically acceptable compositions.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

It is well within the skill of the art to start doses of a nanoparticle at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed nanoparticle and/or composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed nanoparticle and/or composition, by changing the disclosed nanoparticle and/or composition administered, by changing the route of administration, by changing the dosage timing and so on. Dosage can vary, and can be administered in one or more dose administrations daily, for one or several days. In some aspects, doses in the range of 1×10⁶ to 1×10¹⁰ nanoparticles or doses in the range of 1×10⁷ to 1×10⁹ nanoparticles.

The nanoparticles may be administered by inhalation. One skilled in the art would recognize that the nanoparticles in an can be suspended or dissolved appropriate pharmaceutically acceptable carrier and administered, for example, directly into the lungs using a nasal spray or inhalant.

A pharmaceutical composition including the nanoparticle can be administered as an aerosol formulation which contains the nanoparticle in suspended or emulsified form in a propellant or a mixture of solvent and propellant. The aerosolized formulation is then administered through the respiratory system or nasal passages.

An aerosol formulation used for nasal administration is generally an aqueous solution designed to be administered to the nasal passages in drops or sprays. Nasal solutions are generally prepared to be similar to nasal secretions and are generally isotonic and slightly buffered to maintain a pH of about 5.5 to about 6.5, although pH values outside of this range can additionally be used. Antimicrobial agents or preservatives can also be included in the formulation.

The invention further provides methods to enhance the production of FN⁺ matrimeres by cells. In accordance with such methods, a mammalian cell (e.g., an ex vivo human cell or mammalian cell line) is contacted with an effective amount of lysosome activator, e.g., a mucolipin transient receptor potential channel 1 (TRPML1) agonist or transcription factor EB (TFEB) agonist to stimulate or enhance the production of FN+ matrimeres by the mammalian cell. Exemplary lysosome activators include, but are not limited to, ML-SA5 (N′-[3-Chloro-2-(1-piperidinyl)phenyl]-N,N-dimethyl-1,4-benzenedisulfonamide; CAS No. 2418670-70-7), ML-SA1 (2-[2-oxo-2-(2,2,4-trimethyl-1,2,3,4-tetrahydroquinolin-1-yl)ethyl]-2,3-dihydro-1H-isoindole-1,3-dione; CAS No. 332382-54-4), and SF-51 (2-[2-Oxo-2-(2,2,4-trimethyl-1 (2H)-quinolinyl)ethyl]-1H-isoindole-1,3 (2H)-dione), Resveratrol (3,4′,5-trihydroxystilbene), Curcumin analog C1 ((1E,4E)-1,5-bis(2-methoxyphenyl)-1,4-pentadien-3-one; CAS No. 39777-61-2), and Progestin R5020 (CAS No. 34184-77-5).

In addition, the invention provides a method for enabling exogenous protein delivery into phagocytic cells in tissue of a subject by administering to the subject an effective amount of the isolated nanoparticle of the invention which includes an exogenous protein.

Furthermore, the invention provides a method for depositing an exogenous protein in a tissue of a subject by administering to the subject an effective amount of the isolated nanoparticle of the invention which includes an exogenous protein.

The foregoing may be better understood by the following examples, which are presented for purposes of illustration and are not intended to limit the scope of the invention.

### Example 1: Materials and Methods

In vitro Assembly of Matrimeres. Recombinant mouse fibronectin protein and fragments of genomic DNA from mouse mesenchymal stromal cells were used in the assembly of matrimeres. Briefly, genomic DNA was isolated from mammalian cells (e.g., mouse mesenchymal stromal cells) using a commercial DNA extraction kit (e.g., Qiagen). The genomic DNA was sonicated (in 100 ng/μl, 700 μl at a time) with 30% amp for 10 seconds on ice. To autoclaved double filtered 0.1 M sodium acetate and acetic acid buffer solution (8916 μL at pH ˜5.5) was added 84 μl (10 μg/ml final concentration) of recombinant mouse fibronectin (FN) (stock solution 1.2 mg/ml) at 4° C. The mixture was stirred for 10 minutes at 4° C. One mL of fragmented DNA (in 100 ng/μl) was added to the mixture dropwise with continuous stirring (400-500 rpm). Thus, the final concentration of fragmented DNA was 10 ng/μl. The reaction was continued for 12 hours at 4° C. The reaction was either dialyzed against 3.5 kDa or ultracentrifuged at 100,000 g for 90 minutes at 4° C. with 30% sucrose gradient to purify the particles.

Notably, this procedure can be applied to any recombinant (purified or synthesized) matrix protein or peptide that contains a heparin binding domain, along with any isolated negatively charged molecule, e.g., a nucleic acid, synthetic polyphosphate, sulfated polymer (e.g., a sulfated glycosaminoglycan such as heparin sulfate or a sulfated polysaccharide), or polysulfate backbone-based polymer.

The expected yield was 1.5×10¹⁰ particles per ml reaction or per 10 μg/ml fibronectin and 10 ng/μl fragmented genomic DNA. It was observed that 0.75×10⁸ of these particles was sufficient to treat a 20 g mouse after endotoxemia injury.

Enrichment of FN-Bearing Matrimeres Secreted from Cells. Approximately five million cells (e. g., mouse mesenchymal stem cells (MSCs)) were seeded per 175 cm² flask. The cells were cultured in Dulbecco's Modified Eagle Medium (DMEM)/10% premium grade fetal bovine serum (FBS)/1% penicillin/streptomycin/1% glutamine (e.g., GLUTAMAX™) for 24 hours. The cells were subsequently washed once with Hanks' Balanced Salt Solution (HBSS) and then incubated with serum-free DMEM/1% penicillin/streptomycin/1% glutamine for 1 hour. Twenty ml of DMEM with 5% exosome-depleted FBS and 1% penicillin/streptomycin/1% glutamine was added to the cells and the mixture was cultured for 48 hours. The culture medium was collected and serially centrifuged at 2,000 g at 4° C. to remove floating cells/debris and 10,000 g to remove larger particles (>500 nm). Sucrose (30%) was added to the medium and the medium was centrifuged at 100,000 g at 4° C. to remove the soluble portion of the culture medium. Centrifugation was repeated with phosphate-buffered saline (PBS) or HBSS with 30% sucrose to obtain ‘crude’ small extracellular nanostructures (SENS). An additional density gradient centrifugation (e.g., with iodixanol) may optionally be included to further separate sENs into different fractions, which can then be selected to achieve further purification.

The concentration of SENS was determined by nanoparticle tracking analysis (e.g., NanoSight). The expected yield was ˜1000 sENs per cell. Without enrichment, cells secrete ˜33% of FN-bearing matrimeres. After this enrichment procedure, over 70% of FN-bearing matrimeres can be obtained.

The FN-bearing matrimeres may subsequently be enriched by the immunoaffinity-based separation procedures described below. Alternatively, the FN-bearing matrimeres may be enriched by pre-treatment of MSCs with pharmacological agonists for lysosomal functions, such as curcumin analog C1 (CA-C1) (activator of transcription factor EB, which is essential for lysosomal biogenesis) and ML-SA5 (activator of transient receptor potential mucolipin-1, which is a lysosomal cation channel).

Depletion Protocol 1: Antibodies Attached to Magnetic Beads. The magnetic beads were three times with autoclaved double filtered PBS (1×). After the final washing, 333.34 μL of autoclaved double filtered PBS (1×) was added to the beads to make the fresh stock of 4×10⁸ magnetic beads (e.g., DYNABEAD™). Twenty μg of biotinylated-anti-mouse CD63 antibody (or any antibody for any vesicular marker) was added to 4×10⁸ magnetic beads. The mixture was incubated for 1 hour at room temperature with gentle tilting and rotating. The beads were washed with autoclaved double filtered PBS (1×) 4-5 times to remove unbound antibody. The antibody-attached magnetic beads were incubated with sENs (10⁸ to 10⁹ particles) for 1 hour at room temperature (25° C.) with gentle tilting and rotating. After incubation, a magnet was applied to separate the magnetic beads from the solution. The clear solution was collected and centrifuged at 3000 rpm for 2 minutes at 4° C. to remove magnetic beads. The supernatant that contained the enriched matrimeres was collected.

Depletion Protocol 2: Antibodies Attached to Polyethylene Glycol (PEG) Gels. To a solution of streptavidin (1 mg in 370 μl MILLI-Q™ water) was added 30 μl 7.5% Na₂CO₃ (890 μM) solution to maintain the pH at 8. The solution was stirred for 10 minutes at 4° C. One hundred μl of acrylic acid N-hydroxysuccinimide ester (1.35 mg/mL solution freshly prepared in MILLI-Q™ water) was added to the solution and the reaction was continued for 4 hours at 4° C. Fifty μl poly(ethylene glycol) diacrylate solution (density 1.12 mg/mL) and 50 μl of lithium phenyl-2,4,6-trimethylbenzoylphosphinate solution (5 mg/mL solution freshly prepared in MILLI-Q™ water) were added to the solution and the solution was exposed to 365 nm ultraviolet light for 1 minute to polymerize the solution into a gel. The gel was washed three times with autoclaved double filtered PBS (1×) and incubated with 1 μg/ml of biotinylated-anti-mouse CD63 antibody for 1 hour at room temperature (25° C.) with gentle shaking. Free antibody was removed from the gel by washing three times with autoclaved double filtered PBS (1×). The gel was subsequently incubated with sENs (10⁸ to 10⁹ particles) for 1 hour at room temperature (25° C.) with gentle shaking. The supernatant was collected after 1 hour.

Solid Phase Heparin Binding Assay. Solid phase binding assays may be performed to assess extracellular matrix protein (or fragment thereof) binding to heparin-BSA following a previously established protocol. See, e.g., Lin et al. (2002) J. Biol. Chem. 277:50795-50804. Briefly, 10 μg/ml heparin-BSA or extracellular matrix protein (or fragment thereof) in TBS are immobilized per well on a 96-well plate overnight at 4° C. Three 5-min washes with 200 μl of TBS/Ca containing 0.05% Polysorbate 20 are performed between all subsequent steps to remove unbound ligand or antibody. Nonspecific binding surfaces on the plastic are blocked with blocking buffer (5% (w/v) nonfat milk in TBS/Ca) for 1 hour. Serial dilutions (1:2 starting at either 100 μg/ml or at the final concentrations of individual protein pools after gel filtration) of extracellular matrix protein (or fragment thereof) in binding buffer (2% (w/v) nonfat milk in TBS/Ca) are incubated for 2 hours. Polyclonal primary antibody against the extracellular matrix protein (or fragment thereof) is diluted in binding buffer (1:1000) and incubated with the bound soluble ligand for 90 min. Secondary goat anti-rabbit antibody conjugated to horseradish peroxidase is diluted in binding buffer (1:800) and added to the wells for a 90-min incubation. The final color reaction is developed with 5-aminosalicylic acid for 2-20 min, and the reaction is stopped with 2 m sodium hydroxide.

### Example 2: Identification of a FN⁺ Matrimere Subpopulation Distinct from CD63⁺ Extracellular Vesicles

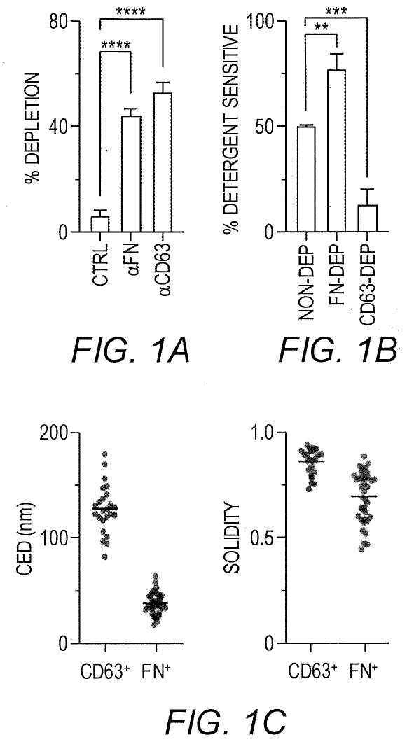

Since FN is a known extracellular matrix (ECM) protein that binds to α₅β₁ and αᵥβ₃ integrins and activates FAK, it was determined which nanostructure subpopulations secreted from mouse bone marrow MSCs contained FN. After removal of larger particles, including apoptotic bodies and microvesicles, ultracentrifugation at 100,000 g results in a pellet composed of bilayer-enclosed extracellular vesicles and non-vesicular nanoparticles. Leveraging an immunoaffinity-based approach, this fraction was further purified based on the expression of FN and the pan-vesicle membrane protein CD63. The gel-based immunoaffinity capture procedures showed that the CD63⁺ fraction was ˜47% and FN⁺ fraction was ˜38% of the total population (FIG. 1A). About 50% of the total population was sensitive to detergent (TRITON™-X)-mediated disruption that removes lipid-bound structures but retains non-lipid particles. Interestingly, ˜77% of FN-depleted fraction was eliminated by detergent-mediated disruption, while ˜13% of CD63-depleted fraction was sensitive to detergent treatment (FIG. 1B). Based on these values, Bayes' theorem could be used to infer that MSC-secreted nanostructures were composed of detergent-sensitive FN⁻CD63⁺ (˜41%) and detergent-resistant FN⁺CD63⁻ (˜33%), wherein the rest of the population was FN⁻CD63⁻ (˜20%) and FN⁺CD63⁺ (˜5%). Thus, the majority of FN⁺ fraction from MSC-secreted nanostructures was a non-vesicular subpopulation, which was enriched by CD63-depletion. To confirm these observations, transmission electron microscopy (TEM) analysis of fractions was performed. Confirmation of nanostructures after positive selection with FN or CD63 antibody is shown in FIG. 1C.

To classify different subpopulations in a non-biased manner, each particle was measured based on 12 morphological parameters and principal component analysis (PCA) was performed to reduce dimensionality, followed by k-means clustering. The identified analysis four distinct subpopulations including (a) spherical particles, (b) non-spherical particles, (c) spherical vesicles and (d) ‘non-spherical’ vesicles. The majority of the FN-depleted group was composed of vesicles, while the CD63-depleted group mostly contained particles without vesicles.

### Example 3: FN⁺ Particles are Naturally Present in Blood Plasma and Sensitive to Inflammatory Injury

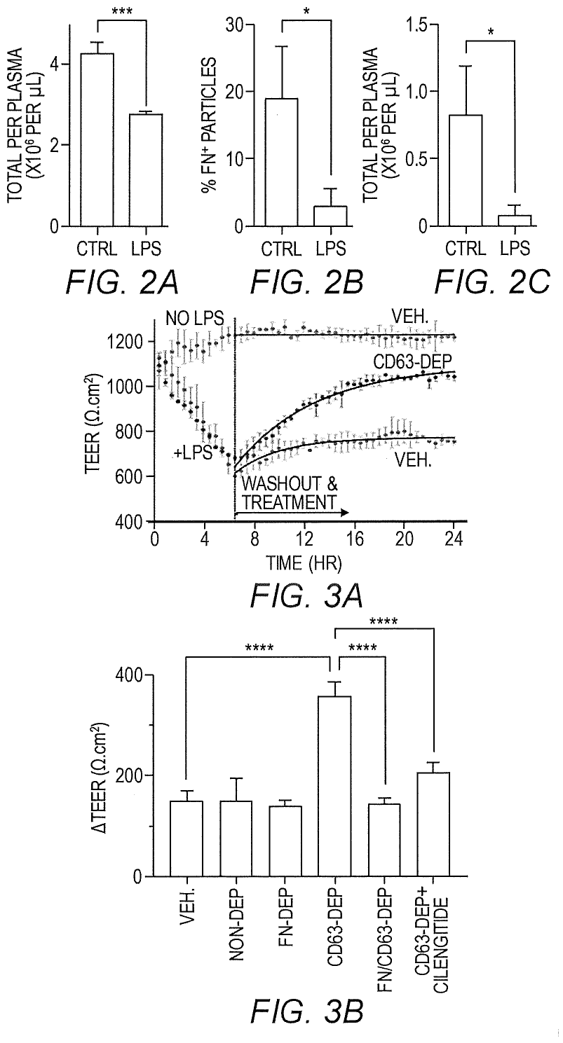

Plasma FN is a major protein component of blood plasma (300 g/ml), which is secreted by hepatocytes in a soluble form. In contrast, cell-secreted FN is known to be present at lower levels (˜1 μg/ml) in blood plasma. However, it is unclear whether nanoscale FN⁺ particles are present in blood plasma, and how their level changes upon tissue injury. Therefore, the number of FN⁺ particles in mouse blood plasma was quantified before and after lipopolysaccharide (LPS) treatment intraperitoneally (i.p.) to induce endotoxemia-mediated vascular hyperpermeability in tissues. There were ˜4 million nanostructures per μl of blood plasma (FIG. 2A), and ˜20% of them were FN⁺ particles (FIG. 2B). Given that blood volume of a mouse is ˜80 μl/g, the results indicated that there were ˜1.3×10⁹ FN⁺ particles in blood per 20 g mouse. Interestingly, LPS treatment not only reduced the total number of nanostructures, but also decreased the fraction of FN⁺ particles, resulting in reduced number of FN⁺ particles in blood by ˜10-fold (FIG. 2C). Thus, the level of endogenous FN⁺ particles in blood plasma is compromised by the injury that disrupts endothelial junctional integrity.

### Example 4: FN⁺ Matrimeres Reverse LPS-Induced Endothelial Hyperpermeability in an RGD-Dependent Manner In Vitro

The effect of depleting different mouse MSC-secreted nanostructure subpopulations on their ability to reverse hyperpermeability of the endothelial cell monolayer after LPS treatment in vitro was assessed by a transendothelial electrical resistance (TEER) assay. TEER was decreased with LPS over 6 hours, while TEER was only partially rescued after washing out LPS in 24 hours. Interestingly, the CD63-depleted (i.e., FN-enriched; FIG. 1A) fraction, but not non-depleted or FN-depleted nanostructures significantly restored TEER compromised by LPS when the same dose (1.5×10⁸ nanostructures) was applied to the endothelial layer (FIG. 3A). Additionally removing FN after CD63 depletion eliminated this effect (FIG. 3B), confirming that FN⁺ matrimeres were essential for rescue of TEER. Competitive inhibition of RGD-binding integrins by cilengitide (cyclic RGD, 200 nM) impaired the ability of CD63-depleted fraction to restore TEER (FIG. 3B). Thus, FN⁺ matrimeres limit endothelial permeability via integrin activation.

### Example 5: FN⁺ Matrimeres Restore LPS-Induced Edema and Vascular Permeability in the Lungs In Vivo

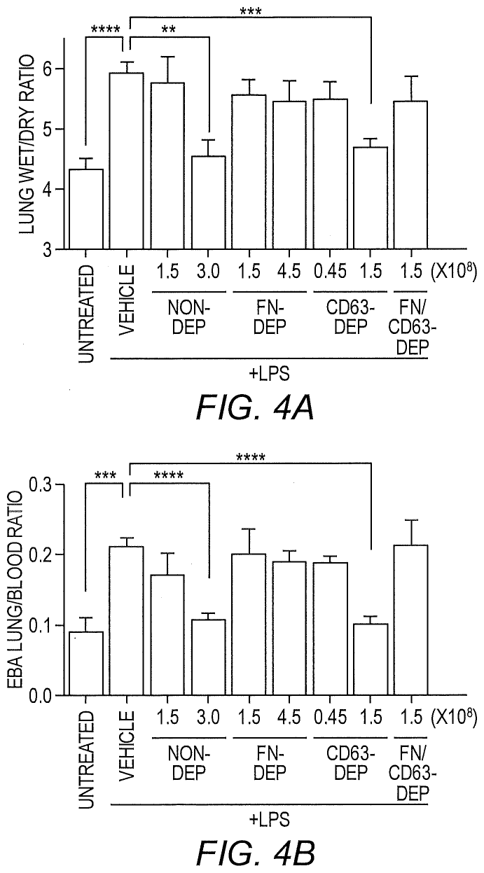

The data herein indicated that FN⁺ matrimere-enriched fraction from MSC-secreted nanostructures could be used to replenish the lost FN⁺ particles in blood plasma (FIGS. 2A-2B) and accelerate the restoration of endothelial barrier function after injury (FIG. 3A-3B). To demonstrate this, mice (10- to 12-week-old C57BL/6 mice) were i.p. treated with LPS (10 mg/ml), followed by treatment with MSC-secreted extracellular nanostructures after 4 hours. MSC-secreted delivered via extracellular nanostructures were the intratracheal (i.t.) route to localize the delivery to the lungs. Mice were analyzed for edema and lung permeability after 24 hours. Without fractionation, 3.0×10⁸ nanostructures per 20 g mouse were necessary to reverse LPS-induced lung edema (FIG. 4A) and vascular permeability (FIG. 4B). In contrast, FN-depleted nanostructures did not show the effects even with a higher dose at 4.5×10⁸. On the other hand, FN⁺ enrichment by CD63-depletion reduced the effective dose to 1.5×10⁸, while removing the FN⁺ fraction after CD63-depletion abolished this effect. Based on the analysis presented herein, ˜1.0×10⁸ FN⁺ matrimeres were needed per 20 g mouse to restore endothelial barrier function in the lungs via the i.t. route. Overall, these results indicated that FN⁺ matrimeres activate endothelial cells and eventually stabilize junctional integrity.

### Example 6: DNA is Needed for the Functionality of FN⁺ Matrimeres

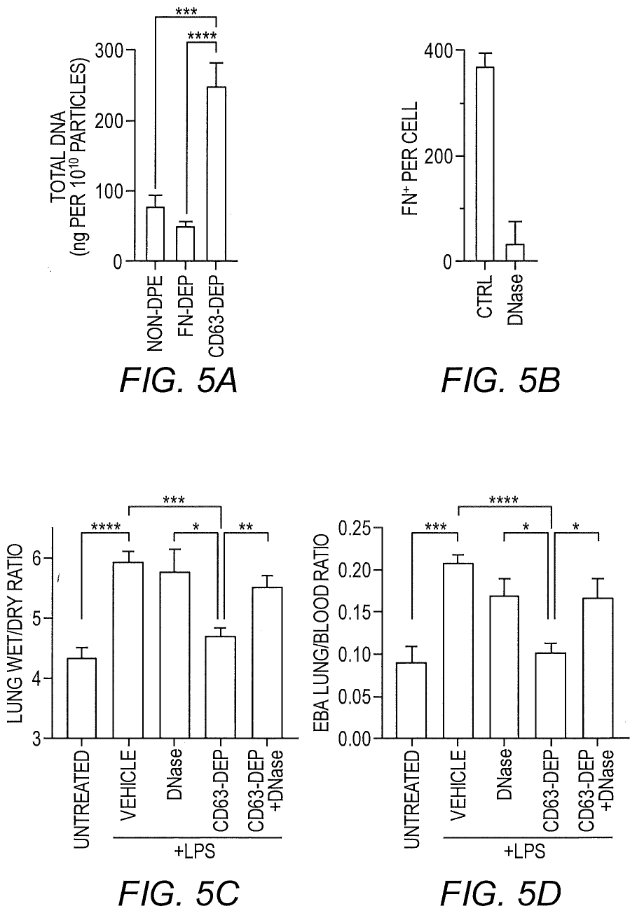

The data presented herein show that enriching EN fraction by CD63-depletion results in ˜3-fold increase in total DNA, indicating that DNA is more abundant in non-vesicular particles (FIG. 5A). DNase I (50 U/ml, 1 hour) treatment of MSC-secreted nanostructures decreased the number of FN⁺ matrimeres (FIG. 5B), indicating that DNA is required for their integrity. It was tested whether DNA is required for the ability of the FN-enriched fraction to restore endothelial barrier function by pre-incubating CD63-depleted fraction from marrow mouse MSCs (1.5×10⁸ per 20 g mouse) with DNase I prior to i.t. delivery into LPS-treated mice. After 24 hours, DNase I alone at this concentration did not have an effect on lung edema (FIG. 5C) or vascular hyperpermeability (FIG. 5D), while DNase I abolished the therapeutic effects of the CD63-depleted fraction. Thus, the results indicate that functionality of FN-enriched particle fraction requires DNA.

### Example 7: Pharmacological Studies Implicate the Role of Autophagosome and Lysosome in Mediating Biogenesis of FN⁺ Matrimeres

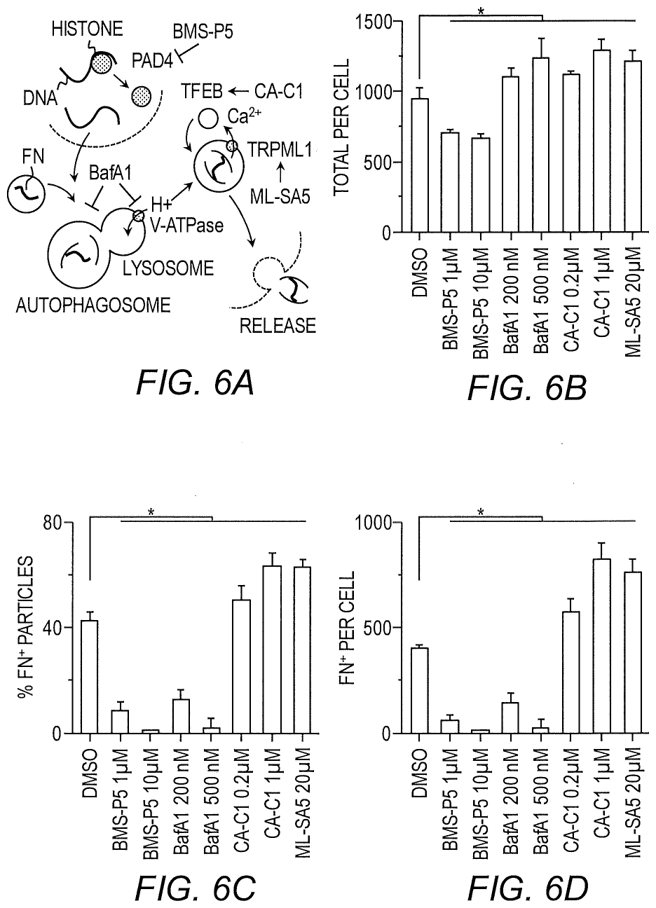

For excess DNA to be released from the nucleus, arginine in histone undergoes modification from positive to neutral charge via citrullination, which is mediated by peptidylarginine deiminase (PAD) (FIG. 6A). The data showed that BMS-P5, a selective inhibitor of PAD4 decreased FN⁺ matrimeres secreted from MSCs (FIG. 6B-D). Interestingly, bafilomycin-A1 (BafA1), which blocks both autophagosome-lysosome fusion and lysosome acidification via vacuolar H⁺ ATPase (FIG. 6A), also decreased MSC-secreted FN⁺ matrimeres (FIG. 6B-D), indicating that these biological processes are likely required for FN⁺ matrimere biogenesis. Conversely, activating components of lysosome function either by ML-SA5 (Ca²⁺ channel TRPML1 agonist) or curcumin analog C1 (CA-C1, TFEB agonist) (FIG. 6A) significantly increased FN⁺ particle number per cell (FIG. 6B-D). Most cells (>90%) remained viable after drug treatment. Thus, autophagy and lysosome are potential mediators of FN⁺ matrimere biogenesis.

### Example 8: FN⁺ Matrimere-Like Particles can be Formed by Recombinant Proteins and Fragmented Genomic DNA

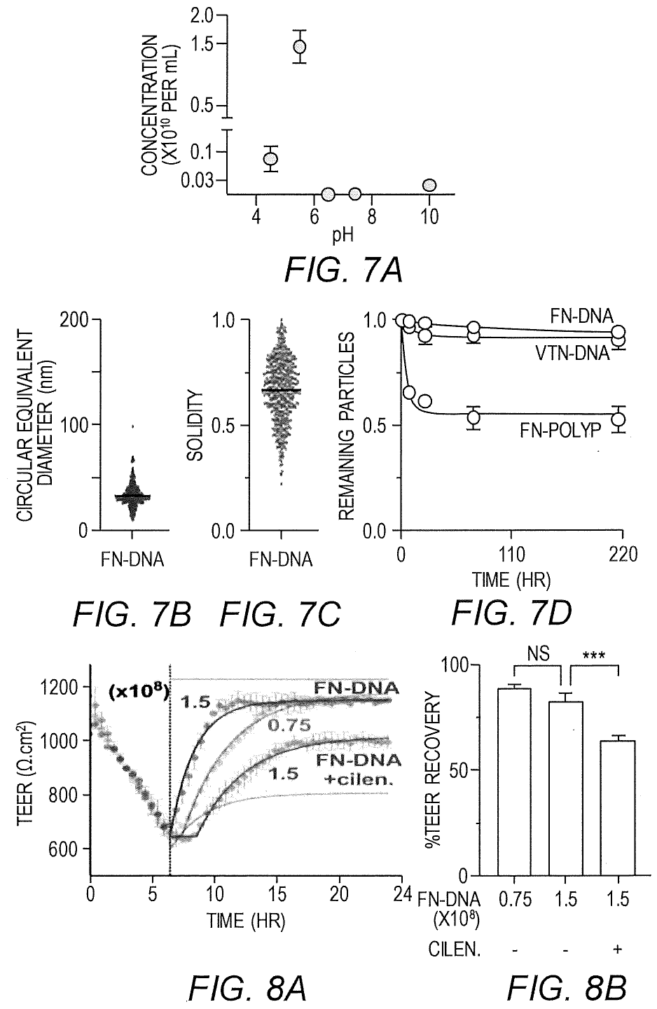

It was subsequently determined whether FN⁺ matrimeres could be reconstituted in vitro. Purified mouse plasma FN (10 μg/ml) was incubated with mouse MSC genomic DNA (10 μg/ml) that was fragmented by sonication, and assembly into nanostructures was assessed. A large number of particles (˜1.5×10¹⁰ per ml) were formed when FN and fragmented DNA were mixed at pH 5.5 as opposed to other tested pHs (FIG. 7A). Incubating the recombinant FNIII₁₂₋₁₃ protein fragment that corresponds to a C-terminal heparin binding domain (Hep-II from human, 181 amino acid residues, 91% homology to mouse; Kerafast) with DNA fragments also resulted in particle formation (˜0.5×10¹⁰ per ml), indicating that this domain is important in the assembly of FN-DNA particles. Principal component analysis showed that reconstituted FN-DNA particles were morphologically similar to non-vesicular particles from MSCs (FIG. 7B-7C). Thus, FN and DNA are sufficient to form nanoparticles at an optimal acidic pH.

In addition to FN and DNA, in vitro matrimere assembly was achieved with vitronectin and DNA, as well as FN and polyphosphate (non-DNA) (FIG. 7D).

### Example 9: Reconstituted FN-DNA Nanoparticles are Sufficient to Resolve Endothelial Hyperpermeability after LPS Treatment In Vitro

It was subsequently determined whether in vitro reconstituted FN-DNA particles could restore permeability of the endothelial cell monolayer after LPS treatment. Adding 1.5×10⁸ FN-DNA particles after a 6-hour LPS treatment rapidly restored TEER with a recovery t1/2 of less than 2 hours (FIG. 8A), which is approximately 3-times more rapid than FN-enriched particles from MSCs (FIG. 3A). Inhibition of RGD-binding integrins by cilengitide (200 nM) partially impaired the ability of reconstituted FN-DNA particles to restore TEER (FIG. 8B). Thus, reconstituted FN-DNA particles are potent agents to reverse endothelial hyperpermeability after LPS treatment in vitro.

### Example 10: Reconstituted Nanoparticles Restore LPS-Induced Edema and Vascular Permeability in the Lungs In Vivo

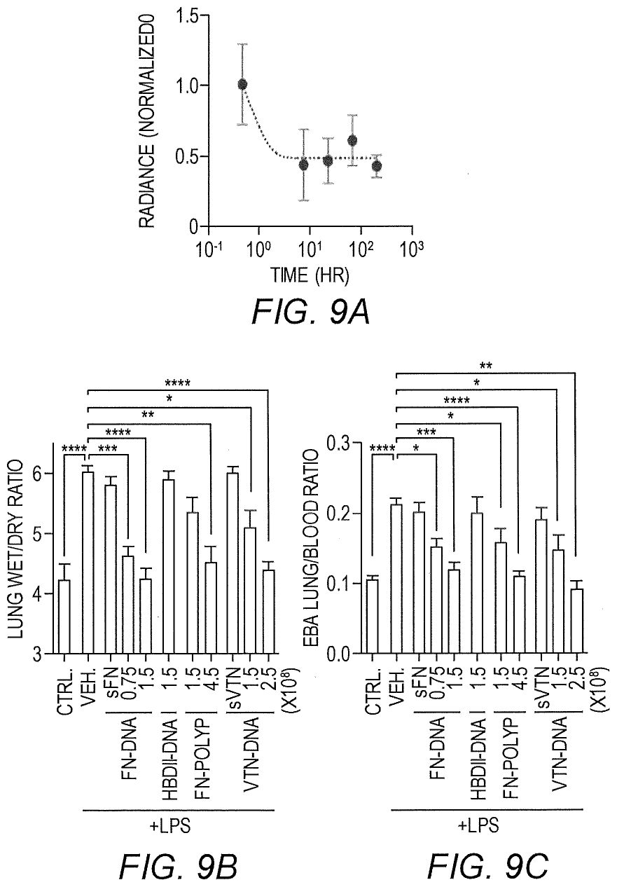

It was determined whether reconstituted particles are sufficient to accelerate the resolution of edema and vascular hyperpermeability after LPS treatment in mice. Reconstituted FN-DNA, VTN-DNA and FN-PolyP particles were labeled with a near-infrared dye Cy7 by amine-reactive (NHS) chemistry to test whether they remained in the lungs of LPS-treated mice after i.t. delivery by IVIS imaging. About 25% of the Cy7 signal remained in the lungs after 8 hours delivery and was sustained for 24 hours (FIG. 9A). FN-DNA, VTN-DNA and FN-PolyP particles were potent in their ability to reverse LPS-induced lung edema (FIG. 9B) and vascular permeability (FIG. 9C), since 0.75×10⁸ particles per 20 g mouse were still sufficient to achieve the therapeutic effects. Overall, these results indicate that reconstituted particles of the invention provide a nanomedicine-based approach to treat tissue injury by restoring endothelial barrier function.

### Example 11: Assembly of FN-Derived Peptides and Genomic DNA Fragments into Particles

The FNIII₁₂₋₁₃ protein fragment that forms particles with fragmented DNA is composed of the arginine rich sequence PPRRARVT (SEQ ID NO: 3), which is essential for recruitment of heparin sulfate. There are three heparin binding domains in FN. Both FN-enriched particles from MSCs and reconstituted FN-DNA particles require RGD (from FNIII₁₀) for their bioactivity (FIG. 3B and FIG. 8B). To this end, test peptide sequences are designed that are composed of PPSRN (SEQ ID NO: 1), RGDSP (SEQ ID NO:2), and three PPRRARVT (SEQ ID NO: 3) domains at different positions (FIG. 10). The total peptide length is kept at 52 amino acid residues with each domain linked by a glycine linker (GGG) (4911.4 g/mol, from Peptide 2.0). Each peptide (0.1 μg/ml, 1 μg/ml, 10 μg/ml) is mixed with sonicated genomic DNA (10 μg/ml) from mouse MSCs at pH 5.5 and incubated at overnight 4° C., followed by ultracentrifugation. After resuspending in PBS, nanoparticle tracking analysis (NanoSight) is used to quantify particle concentration, while TEM is used to analyze morphology. The effect of different particle formulations (1.5×10⁸) on rescue of TEER in primary lung endothelial cells after LPS treatment is determined (FIG. 8A-8B).

### Example 12: Assembly of Purified FN and Synthetic DNA into Particles

The data herein show that most fragmented DNAs are between 600-1000 bp in size. Thus, double-stranded DNA (dsDNA) fragments composed of a 250 bp random sequence unit with 50% GC content are generated. Gene fragments with varied length are synthesized by repeating the 250 bp random sequence unit to generate 250, 500, 750, 1000, 1250, 2000, 2500, 5000, 7500 or 10000 bp fragments. After screening for the DNA length(s) that results in the greatest number of particles and confirming the size(s) of the same, the sequence unit is modified to vary GC content (e.g., 40%, 50%, or 60% GC content) and the effect on particle formation is determined. The endothelial TEER assays shown in FIG. 8A-8B are used to ascertain the functionality of different particle formulations in reversing LPS-induced hyperpermeability in vitro.

### Example 13: Role of Synthetic Matrimeres in Restoring Endothelial Barrier Function after LPS Injury In Vivo

The assembly of particles from a selected pair of FN-derived peptide and DNA sequence as described above is tested for its ability to restore TEER after LPS treatment in vitro. After 4 hours i.p. LPS treatment, i.t. delivery of Cy7-NHS functionalized (and dialyzed) particles (1.5×10⁸ per 20 g mouse) is used to track biodistribution of the particles over 7 days by IVIS imaging as in FIG. 9A. Soluble Cy7 is used as a control. Lungs are also isolated to quantify the localization of synthetic particles in collagen fibers vs. CD31⁺ vasculature by two-photon imaging, followed by analysis using Imaris or ImageJ. Dose-dependent studies (0.015×10⁸, 0.05×10⁸, 0.15×10⁸, 0.5×10⁸, 1.5×10⁸ per 20 g mouse) are also performed to define the potency (half-maximum dose) in which synthetic particles resolve lung edema and vascular permeability after 24 hours of i.t. delivery into LPS-treated mice. The peptide and the DNA are also tested separately in a soluble form as controls. To investigate whether therapeutic effects of synthetic matrimeres can also be observed with different routes of administration, i. v. injection (retro-orbital) and inhalation of the aerosolized particles are tested. For aerosolization, a dose range of 0.015×10⁸ to 1.5×10¹⁰ particles (5 doses) per ml PBS is delivered to 4 mice at a time.

What is claimed is:

1. An isolated nanoparticle comprising
(i) at least one extracellular matrix protein or fragment thereof, the at least one extracellular matrix protein or fragment thereof comprising at least one heparin binding domain; and
(ii) nucleic acid molecules or polyphosphate molecules comprising about 30 to about 10000 nucleotides or phosphate units.

2. The isolated nanoparticle of claim 1, wherein the at least one extracellular matrix protein further comprises at least one cell adhesion sequence.

3. The isolated nanoparticle of claim 1, wherein the at least one extracellular matrix protein comprises fibronectin, fibrinogen/fibrin, vitronectin, laminin, collagen, fibulin, fibrillin, or tropoelastin, or a combination thereof.

4. The isolated nanoparticle of claim 1, wherein the nucleic acid molecules comprise fragmented genomic DNA from mammalian cells.

5. The isolated nanoparticle of claim 1, wherein the nanoparticles are isolated from plasma.

6. The isolated nanoparticle of claim 1, wherein the nanoparticles are isolated from cell culture medium.

7. The isolated nanoparticle of claim 1, wherein the nanoparticles are reconstituted from at least one isolated or recombinant extracellular matrix protein and isolated nucleic acid molecules from mammalian cells.

8. The isolated nanoparticle of claim 1, wherein the nanoparticles are reconstituted from at least one isolated or recombinant extracellular matrix protein and synthetic nucleic acid molecules.

9. The isolated nanoparticle of claim 1, wherein the nanoparticles are reconstituted from at least one isolated or recombinant extracellular matrix protein and polyphosphate molecules.

10. The isolated nanoparticle of claim 1, wherein the nanoparticles are reconstituted from at least one synthetic extracellular matrix-derived peptide and isolated nucleic acid molecules from mammalian cells.

11. The isolated nanoparticle of claim 1, wherein the nanoparticles are reconstituted from at least one synthetic extracellular matrix-derived peptide and synthetic nucleic acid molecules.

12. The isolated nanoparticle of claim 1, wherein the nanoparticles are reconstituted from at least one synthetic extracellular matrix-derived peptide and polyphosphate molecules.

13. A composition comprising the isolated nanoparticle of claim 1 and a pharmaceutically acceptable carrier or diluent.

14. A method of enhancing or restoring endothelial barrier function in a subject comprising administering to a subject an effective amount of the isolated nanoparticle of claim 1 thereby enhancing or restoring endothelial barrier function in the subject.

15. A method of treating tissue injury in a subject comprising administering to the subject an effective amount of the isolated nanoparticle of claim 1 thereby treating tissue injury in the subject.

16. The method of claim 15, wherein the tissue injury comprises vascular hyperpermeability, edema, or a combination thereof.

17. The method of claim 15, wherein the tissue injury comprises tissue damage, inflammation, degeneration, fibrosis, or a combination thereof.

18. A method for preparing the nanoparticle of claim 1 comprising contacting, at a pH in the range of about 4 to 6, the at least one extracellular matrix protein or fragment thereof with the nucleic acid molecules or polyphosphate molecules thereby preparing the nanoparticle.


SEQUENCE LISTING (53 sequences)

SEQ ID NO: 1 - protein, 5 aa, Homo sapiens
PPSRN

SEQ ID NO: 2 - protein, 5 aa, Homo sapiens
RGDSP

SEQ ID NO: 3 - protein, 8 aa, Homo sapiens
PPRRARVT

SEQ ID NO: 4 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 5 - protein, 8 aa, Homo sapiens
WQPPRARI

SEQ ID NO: 6 - protein, 6 aa, Homo sapiens
PPRARV

SEQ ID NO: 7 - protein, 6 aa, Homo sapiens
PRRGRV

SEQ ID NO: 8 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 9 - protein, 28 aa, Homo sapiens
GHRPLDKKREEAPSLRPAPPPISGGGYR

SEQ ID NO: 10 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 11 - protein, 12 aa, Homo sapiens
KKQRFRHRNRKG

SEQ ID NO: 12 - protein, 3075 aa, Homo sapiens
MRGGVLLVLLLCVAAQCRQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGMCICYGHASSCPWDETTKKLQCQCEHNTCGESCNRCCPGYHQQPWRPGTVSSGNTCEACNCHNKAKDCYYDESVAKQKKSLNTAGQFRGGGVCINCLQNTMGINCETCIDGYYRPHKVSPYEDEPCRPCNCDPVGSLSSVCIKDDLHSDLHNGKQPGQCPCKEGYTGEKCDRCQLGYKDYPTCVSCGCNPVGSASDEPCTGPCVCKENVEGKACDRCKPGFYNLKEKNPRGCSECFCFGVSDVCSSLSWPVGQVNSMSGWLVTDLISPRKIPSQQDALGGRHQVSINNTAVMQRLAPKYYWAAPEAYLGNKLTAFGGFLKYTVSYDIPVETVDSNLMSHADVIIKGNGLTLSTQAEGLSLQPYEEYLNVVRLVPENFQDFHSKRQIDRDQLMTVLANVTHLLIRANYNSAKMALYRLESVSLDIASSNAIDLVVAADVEHCECPQGYTGTSCESCLSGYYRVDGILFGGICQPCECHGHAAECNVHGVCIACAHNTTGVHCEQCLPGFYGEPSRGTPGDCQPCACPLTIASNNFSPTCHLNDGDEVVCDWCAPGYSGAWCERCADGYYGNPTVPGESCVPCDCSGNVDPSEAGHCDSVTGECLKCLGNTDGAHCERCADGFYGDAVTAKNCRACECHVKGSHSAVCHLETGLCDCKPNVTGQQCDQCLHGYYGLDSGHGCRPCNCSVAGSVSDGCTDEGQCHCVPGVAGKRCDRCAHGFYAYQDGSCTPCDCPHTQNTCDPETGECVCPPHTQGVKCEECEDGHWGYDAEVGCQACNCSLVGSTHHRCDVVTGHCQCKSKFGGRACDQCSLGYRDFPDCVPCDCDLRGTSGDACNLEQGLCGCVEETGACPCKENVFGPQCNECREGTFALRADNPLGCSPCFCSGLSHLCSELEDYVRTPVTLGSDQPLLRVVSQSNLRGTTEGVYYQAPDFLLDAATVRQHIRAEPFYWRLPQQFQGDQLMAYGGKLKYSVAFYSLDGVGTSNFEPQVLIKGGRIRKQVIYMDAPAPENGVRQEQEVAMRENFWKYFNSVSEKPVTREDFMSVLSDIEYILIKASYGQGLQQSRISDISMEVGRKAEKLHPEEEVASLLENCVCPPGTVGFSCQDCAPGYHRGKLPAGSDRGPRPLVAPCVPCSCNNHSDTCDPNTGKCLNCGDNTAGDHCDVCTSGYYGKVTGSASDCALCACPHSPPASFSPTCVLEGDHDFRCDACLLGYEGKHCERCSSSYYGNPQTPGGSCQKCDCNPHGSVHGDCDRTSGQCVCRLGASGLRCDECEPRHILMETDCVSCDDECVGVLLNDLDEIGDAVLSLNLTGIIPVPYGILSNLENTTKYLQESLLKENMQKDLGKIKLEGVAEETDNLQKKLTRMLASTQKVNRATERIFKESQDLAIAIERLQMSITEIMEKTTLNQTLDEDFLLPNSTLQNMQQNGTSLLEIMQIRDFTQLHQNATLELKAAEDLLSQIQENYQKPLEELEVLKEAASHVLSKHNNELKAAEALVREAEAKMQESNHLLLMVNANLREFSDKKLHVQEEQNLTSELIVQGRGLIDAAAAQTDAVQDALEHLEDHQDKLLLWSAKIRHHIDDLVMHMSQRNAVDLVYRAEDHAAEFQRLADVLYSGLENIRNVSLNATSAAYVHYNIQSLIEESEELARDAHRTVTETSLLSESLVSNGKAAVQRSSRFLKEGNNLSRKLPGIALELSELRNKTNRFQENAVEITRQTNESLLILRAIPKGIRDKGAKTKELATSASQSAVSTLRDVAGLSQELLNTSASLSRVNTTLRETHQLLQDSTMATLLAGRKVKDVEIQANLLFDRLKPLKMLEENLSRNLSEIKLLISQARKQAASIKVAVSADRDCIRAYQPQISSTNYNTLTLNVKTQEPDNLLFYLGSSTASDFLAVEMRRGRVAFLWDLGSGSTRLEFPDFPIDDNRWHSIHVARFGNIGSLSVKEMSSNQKSPTKTSKSPGTANVLDVNNSTLMFVGGLGGQIKKSPAVKVTHFKGCLGEAFLNGKSIGLWNYIEREGKCRGCFGSSQNEDPSFHFDGSGYSVVEKSLPATVTQIIMLFNTFSPNGLLLYLGSYGTKDFLSIELFRGRVKVMTDLGSGPITLLTDRRYNNGTWYKIAFQRNRKQGVLAVIDAYNTSNKETKQGETPGASSDLNRLDKDPIYVGGLPRSRVVRRGVTTKSFVGCIKNLEISRSTFDLLRNSYGVRKGCLLEPIRSVSFLKGGYIELPPKSLSPESEWLVTFATTNSSGIILAALGGDVEKRGDREEAHVPFFSVMLIGGNIEVHVNPGDGTGLRKALLHAPTGTCSDGQAHSISLVRNRRIITVQLDENNPVEMKLGTLVESRTINVSNLYVGGIPEGEGTSLLTMRRSFHGCIKNLIFNLELLDFNSAVGHEQVDLDTCWLSERPKLAPDAEDSKLLPEPRAFPEQCVVDAALEYVPGAHQFGLTQNSHFILPFNQSAVRKKLSVELSIRTFASSGLIYYMAHQNQADYAVLQLHGGRLHFMFDLGKGRTKVSHPALLSDGKWHTVKTDYVKRKGFITVDGRESPMVTVVGDGTMLDVEGLFYLGGLPSQYQARKIGNITHSIPACIGDVTVNSKQLDKDSPVSAFTVNRCYAVAQEGTYFDGSGYAALVKEGYKVQSDVNITLEFRTSSQNGVLLGISTAKVDAIGLELVDGKVLFHVNNGAGRITAAYEPKTATVLCDGKWHTLQANKSKHRITLIVDGNAVGAESPHTQSTSVDTNNPIYVGGYPAGVKQKCLRSQTSFRGCLRKLALIKSPQVQSFDFSRAFELHGVFLHSCPGTES

SEQ ID NO: 13 - protein, 21 aa, Homo sapiens
KEGYKVRLDLNITLEFRTTSK

SEQ ID NO: 14 - protein, 17 aa, Homo sapiens
KATPMLKMRTSFHGCIK

SEQ ID NO: 15 - protein, 5 aa, Homo sapiens
IKLLI

SEQ ID NO: 16 - protein, 14 aa, Homo sapiens
KDFLSIELVRGRVK

SEQ ID NO: 17 - protein, 1668 aa, Homo sapiens
MPPAVRRSACSMGWLWIFGAALGQCLGYSSQQQRVPFLQPPGQSQLQASYVEFRPSQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHMLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGGPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 18 - protein, 20 aa, Homo sapiens
PPFLMLLKGSTRFNKTKTFR

SEQ ID NO: 19 - protein, 25 aa, Homo sapiens
RLVFALGTDGKKLRIKSKEKCNDGK

SEQ ID NO: 20 - protein, 1823 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGMDCPTISCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 21 - protein, 27 aa, Homo sapiens
PLFLLHKKGKNLSKPKASQNKKGGKSK

SEQ ID NO: 22 - protein, 23 aa, Homo sapiens
TLFLAHGRLVYMFNVGHKKLKIR

SEQ ID NO: 23 - protein, 3695 aa, Homo sapiens [sequence omitted]

SEQ ID NO: 24 - protein, 31 aa, Homo sapiens
TPGLGPRGLQATARKASRRSRQPARHPACML

SEQ ID NO: 25 - protein, 20 aa, Homo sapiens
RQRSRPGRWHKVSVRWEKNR

SEQ ID NO: 26 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 27 - protein, 6 aa, Homo sapiens
KGHRGF

SEQ ID NO: 28 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 29 - protein, 6 aa, Homo sapiens
KGIRGH

SEQ ID NO: 30 - protein, 448 aa, Homo sapiens
MPGIKRILTVTILALCLPSPGNAQAQCTNGFDLDRQSGQCLDIDECRTIPEACRGDMMCVNQNGGYLCIPRTNPVYRGPYSNPYSTPYSGPYPAAAPPLSAPNYPTISRPLICRFGYQMDESNQCVDVDECATDSHQCNPTQICINTEGGYTCSCTDGYWLLEGQCLDIDECRYGYCQQLCANVPGSYSCTCNPGFTLNEDGRSCQDVNECATENPCVQTCVNTYGSFICRCDPGYELEEDGVHCSDMDECSFSEFLCQHECVNQPGTYFCSCPPGYILLDDNRSCQDINECEHRNHTCNLQQTCYNLQGGFKCIDPIRCEEPYLRISDNRCMCPAENPGCRDQPFTILYRDMDVVSGRSVPADIFQMQATTRYPGAYYIFQIKSGNEGREFYMRQTGPISATLVMTRPIKGPREIQLDLEMITVNTVINFRGSSVIRLRIYVSQYPF

SEQ ID NO: 31 - protein, 476 aa, Homo sapiens
QDTEETITYTQCTDGYEWDPVRQQCKDIDECDIVPDACKGGMKCVNHYGGYLCLPKTAQIIVNNEQPQQETQPAEGTSGATTGVVAASSMATSGVLPGGGFVASAAAVAGPEMQTGRNNFVIRRNPADPQRIPSNPSHRIQCAAGYEQSEHNVCQDIDECTAGTHNCRADQVCINLRGSFACQCPPGYQKRGEQCVDIDECTIPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYQVVRSRTCQDINECETTNECREDEMCWNYHGGFRCYPRNPCQDPYILTPENRCVCPVSNAMCRELPQSIVYKYMSIRSDRSVPSDIFQIQATTIYANTINTFRIKSGNENGEFYLRQTSPVSAMLVLVKSLSGPREHIVDLEMLTVSSIGTFRTSSVLRLTIIVGPFSF

SEQ ID NO: 32 - protein, 443 aa, Homo sapiens
MLPCASCLPGSLLLWALLLLLLGSASPQDSEEPDSYTECTDGYEWDPDSQHCRDVNECLTIPEACKGEMKCINHYGGYLCLPRSAAVINDLHGEGPPPPVPPAQHPNPCPPGYEPDDQDSCVDVDECAQALHDCRPSQDCHNLPGSYQCTCPDGYRKIGPECVDIDECRYRYCQHRCVNLPGSFRCQCEPGFQLGPNNRSCVDVNECDMGAPCEQRCFNSYGTFLCRCHQGYELHRDGFSCSDIDECSYSSYLCQYRCINEPGRFSCHCPQGYQLLATRLCQDIDECESGAHQCSEAQTCVNFHGGYRCVDTNRCVEPYIQVSENRCLCPASNPLCREQPSSIVHRYMTITSERSVPADVFQIQATSVYPGAYNAFQIRAGNSQGDFYIRQINNVSAMLVLARPVTGPREYVLDLEMVTMNSLMSYRASSVLRLTVFVGAYTF

SEQ ID NO: 33 - protein, 416 aa, Homo sapiens
QDSEEPDSYTECTDGYEWDPDSQHCRDVNECLTIPEACKGEMKCINHYGGYLCLPRSAAVINDLHGEGPPPPVPPAQHPNPCPPGYEPDDQDSCVDVDECAQALHDCRPSQDCHNLPGSYQCTCPDGYRKIGPECVDIDECRYRYCQHRCVNLPGSFRCQCEPGFQLGPNNRSCVDVNECDMGAPCEQRCFNSYGTFLCRCHQGYELHRDGFSCSDIDECSYSSYLCQYRCINEPGRFSCHCPQGYQLLATRLCQDIDECESGAHQCSEAQTCVNFHGGYRCVDTNRCVEPYIQVSENRCLCPASNPLCREQPSSIVHRYMTITSERSVPADVFQIQATSVYPGAYNAFQIRAGNSQGDFYIRQINNVSAMLVLARPVTGPREYVLDLEMVTMNSLMSYRASSVLRLTVFVGAYTF

SEQ ID NO: 34 - protein, 448 aa, Homo sapiens; identical to SEQ ID NO: 30

SEQ ID NO: 35 - protein, 425 aa, Homo sapiens
QAQCTNGFDLDRQSGQCLDIDECRTIPEACRGDMMCVNQNGGYLCIPRTNPVYRGPYSNPYSTPYSGPYPAAAPPLSAPNYPTISRPLICRFGYQMDESNQCVDVDECATDSHQCNPTQICINTEGGYTCSCTDGYWLLEGQCLDIDECRYGYCQQLCANVPGSYSCTCNPGFTLNEDGRSCQDVNECATENPCVQTCVNTYGSFICRCDPGYELEEDGVHCSDMDECSFSEFLCQHECVNQPGTYFCSCPPGYILLDDNRSCQDINECEHRNHTCNLQQTCYNLQGGFKCIDPIRCEEPYLRISDNRCMCPAENPGCRDQPFTILYRDMDVVSGRSVPADIFQMQATTRYPGAYYIFQIKSGNEGREFYMRQTGPISATLVMTRPIKGPREIQLDLEMITVNTVINFRGSSVIRLRIYVSQYPF

SEQ ID NO: 36 - protein, 2871 aa, Homo sapiens [sequence omitted]

SEQ ID NO: 37 - protein, 168 aa, Homo sapiens
DIDECRISPDLCGRGQCVNTPGDFECKCDEGYESGFMMMKNCMDIDECQRDPLLCRGGVCHNTEGSYRCECPPGHQLSPNISACIDINECELSAHLCPNGRCVNLIGKYQCACNPGYHSTPDRLFCVDIDECSIMNGGCETFCTNSEGSYECSCQPGFALMPDQRSCT

SEQ ID NO: 38 - protein, 119 aa, Homo sapiens
DMRRSLCYRNYYADNQTCDGELLFNMTKKMCCCSYNIGRAWNKPCEQCPIPSTDEFATLCGSQRPGFVIDIYTGLPVDIDECREIPGVCENGVCINMVGSFRCECPVGFFYNDKLLVCE

SEQ ID NO: 39 - protein, 724 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 40 - protein, 698 aa, Homo sapiens
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 41 - protein, 4 aa, Homo sapiens
RGDS

SEQ ID NO: 42 - protein, 5 aa, Homo sapiens
RGDSP

SEQ ID NO: 43 - protein, 6 aa, Homo sapiens
RGDSPK

SEQ ID NO: 44 - protein, 5 aa, Homo sapiens
RGDTP

SEQ ID NO: 45 - protein, 10 aa, Homo sapiens
RGDSPASSKP

SEQ ID NO: 46 - protein, 9 aa, Homo sapiens
GGGGRGDSP

SEQ ID NO: 47 - protein, 6 aa, Homo sapiens
KQAGDV

SEQ ID NO: 48 - protein, 5 aa, Homo sapiens
PHSRN

SEQ ID NO: 49 - protein, 5 aa, Homo sapiens
PPSRN

SEQ ID NO: 50 - protein, 5 aa, Homo sapiens
YIGSR

SEQ ID NO: 51 - protein, 13 aa, Homo sapiens
RLVSYNGIIFFLK

SEQ ID NO: 52 - protein, 4 aa, Homo sapiens
DGEA

SEQ ID NO: 53 - protein, 4 aa, Homo sapiens
VAPG